(19) United States
(12) Patent Application Publication
(10) Pub. No.: US 2014/0017226 A1
(43) Pub. Date: Jan. 16, 2014

(54) TREATMENT OF MULTIPLE SCLEROSIS WITH COMBINATION OF LAQUINIMOD AND FAMPRIDINE

(71) Applicants: Joel Kaye, Netanya (IL); Nora Tarcic, Modiin (IL)

(72) Inventors: Joel Kaye, Netanya (IL); Nora Tarcic, Modiin (IL)

(73) Assignee: TEVA PHARMACEUTICAL INDUSTRIES, LTD., Petach-Tikva (IL)

(21) Appl. No.: 13/939,306

(22) Filed: Jul. 11, 2013

Related U.S. Application Data
(60) Provisional application No. 61/670,758, filed on Jul. 12, 2012.

(51) Int. Cl. A61K 31/4704; A61K 45/06; A61K 31/4409

(52) U.S. Cl. CPC A61K 31/4704; A61K 31/4409; A61K 45/06
USPC 424/130.1; 514/312; 514/161; 514/171

(57) ABSTRACT

This invention provides a method of treating a subject afflicted with multiple sclerosis or presenting a clinically isolated syndrome comprising administering to the subject fampridine as an add-on therapy to or in combination with laquinimod. This invention also provides a package comprising laquinimod and fampridine for treating a subject afflicted with multiple sclerosis or presenting a clinically isolated syndrome. This invention also provides fampridine for use as an add-on therapy or in combination with laquinimod in treating a subject afflicted with multiple sclerosis or presenting a clinically isolated syndrome. This invention also provides a pharmaceutical composition comprising laquinimod and fampridine for use in treating a subject afflicted with multiple sclerosis or presenting a clinically isolated syndrome. This invention further provides use of laquinimod and fampridine in the preparation of a combination for treating a subject afflicted with multiple sclerosis or presenting a clinically isolated syndrome.

This application claims benefit of U.S. Provisional Application No. 61/670,758, filed Jul. 12, 2012, the entire content of which is hereby incorporated by reference herein.

Throughout this application, various publications are referred to by first author and year of publication. Full citations for these publications are presented in a References section immediately before the claims. Disclosures of the documents and publications referred to herein are hereby incorporated in their entireties by reference into this application.

### BACKGROUND

Multiple Sclerosis (MS) is a neurological disease affecting more than 1 million people worldwide. It is the most common cause of neurological disability in young and middle-aged adults and has a major physical, psychological, social and financial impact on subjects and their families, friends and bodies responsible for health care (EMEA Guideline, 2006).

It is generally assumed that MS is mediated by some kind of autoimmune process possibly triggered by infection and superimposed upon a genetic predisposition. It is a chronic inflammatory condition that damages the myelin of the Central Nervous System (CNS). The pathogenesis of MS is characterized by the infiltration of autoreactive T-cells from the circulation directed against myelin antigens into the CNS (Bjartmar, 2002). In addition to the inflammatory phase in MS, axonal loss occurs early in the course of the disease and can be extensive over time, leading to the subsequent development of progressive, permanent, neurologic impairment and, frequently, severe disability (Neuhaus, 2003). Symptoms associated with the disease include fatigue, spasticity, ataxia, weakness, bladder and bowel disturbances, sexual dysfunction, pain, tremor, paroxysmal manifestations, visual impairment, psychological problems and cognitive dysfunction (EMEA Guideline, 2006). MS disease activity can be monitored by cranial scans, including magnetic resonance imaging (MRI) of the brain, accumulation of disability, as well as rate and severity of relapses. The diagnosis of clinically definite MS as determined by the Poser criteria (Poser, 1983) requires at least two neurological events suggesting demyelination in the CNS separated in time and in location. A clinically isolated syndrome (CIS) is a single monosymptomatic attack suggestive of MS, such as optic neuritis, brain stem symptoms, and partial myelitis. Patients with CIS that experience a second clinical attack are generally considered to have clinically definite multiple sclerosis (CDMS). Over 80 percent of patients with a CIS and MRI lesion go on to develop MS, while approximately 20 percent have a self-limited process (Brex, 2002; Frohman, 2003).

Various MS disease stages and/or types are described in Multiple Sclerosis Therapeutics (Duntiz, 1999). Among them, relapsing remitting multiple sclerosis (RRMS) is the most common form at the time of initial diagnosis. Many subjects with RRMS have an initial relapsing-remitting course for 5-15 years, which then advances into the secondary progressive MS (SPMS) disease course. Relapses result from inflammation and demyelination, whereas restoration of nerve conduction and remission is accompanied by resolution of inflammation, redistribution of sodium channels on demyelinated axons and remyelination (Neuhaus, 2003; Noseworthy, 2000).

In April 2001, an international panel in association with the National MS Society of America recommended diagnostic criteria for multiple sclerosis. These criteria became known as the McDonald Criteria. The McDonald Criteria make use of MRI techniques and are intended to replace the Poser Criteria and the older Schumacher Criteria (McDonald, 2001). The McDonald Criteria was revised in March 2005 by an international panel (Polman, 2005) and updated again in 2010 (Polman, 2011).

Intervention with disease-modifying therapy at relapsing stages of MS is suggested to reduce and/or prevent accumulating neurodegeneration (Hohlfeld, 2000; De Stefano, 1999). There are currently a number of disease-modifying medications approved for use in relapsing MS (RMS), which includes RRMS and SPMS (The Disease Modifying Drug Brochure, 2006). These include interferon beta 1-a (Avonex® and Rebif®), interferon beta 1-b (Betaseron®), glatiramer acetate (Copaxone®), mitoxantrone (Novantrone®), natalizumab (Tysabri®) and fampridine (Gilenya®). Most of them are believed to act as immunomodulators. Mitoxantrone and natalizumab are believed to act as immunesuppressants. However, the mechanisms of action of each have been only partly elucidated. Immunosuppressants or cytotoxic agents are used in some subjects after failure of conventional therapies. However, the relationship between changes of the immune response induced by these agents and the clinical efficacy in MS is far from settled (EMEA Guideline, 2006).

Other therapeutic approaches include symptomatic treatment which refers to all therapies applied to improve the symptoms caused by the disease (EMEA Guideline, 2006) and treatment of acute relapses with corticosteroids. While steroids do not affect the course of MS over time, they can reduce the duration and severity of attacks in some subjects.

### Fampridine

Fampridine (4-aminopyridin; 4-AP) is a derivative of pyridine with an amino substitution in the 4-position. It is a basic compound with a molecular structure of C₅H₆N₂, formula weight of 94.12, a melting point of 155-158° C., a boiling point of 273° C. and a pK of 9.18. Its chloride salt is readily soluble in aqueous solution, making it suitable for oral administration. In its unionized form it is lipid-soluble and therefore able to cross the blood-brain barrier, enabling it to interact with channels in the CNS and to cross cell membranes to bind to sites accessible on the cytoplasmic side (Bever and Judge, 2009).

fampridine chemical structure is shown below:

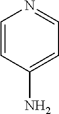

IUPAC: pyridin-4-amine. Other names of fampridine include 4-Pyridinamine, fampridine, 4-Pyridylamine, pyridin-4-amine, Avitrol, p-Aminopyridine, Pyridine, 4-amino-, 4-AP, 504-24-5 (http://pubchem.ncbi.nlm.nih.gov)

AMPYRA® (Dalfampridine; Acorda Therapeutics) Extended Release Tablets containing 10 mg fampridine has been approved by the U.S. Food and Drug Administration (FDA) on Jan. 22, 2010 as a treatment to improve walking in patients with multiple sclerosis (MS). The Generic name of the sustained release 4-aminopyridine (4-AP) is fampridine-SR (slow release; also called fampridine-ER or fampridine-PR). The Recommended dose is one tablet of AMPYRA® two times each day (b.i.d) 1 about 12 hours apart (20 mg/day) (FDA News Release, 2012).

Fampridine has safety issues related to seizure induction (Burton, 2008) Trials of immediate-release fampridine documented a range of side effects and adverse events. It has been found that adverse events were related to peak serum levels while efficacy was related to total drug exposure. This finding led to the development of fampridine SR. However, seizures as well as kidney and bladder infections remain to be serious adverse events associated with the approved drug (Burton, 2008, FDA News Release, 2012; The Ampyra® website).

In light of the safety issues associated with AMPYRA®, the Food and Drug Administration (FDA) required that information about AMPYRA® needs to be communicated to healthcare providers and patients, including “Informing patients about the serious risks associated with use of AMPYRA®”. The patient Medication Guide includes the instruction to: take AMPYRA® “exactly as the doctor tells you”; not to change the dose of AMPYRA®; to take one tablet of AMPYRA® 2 times each day about 12 hours apart; not to take more than 2 tablets of AMPYRA® in a 24-hour period; to take AMPYRA® tablets whole—not to break, crush, chew or dissolve AMPYRA® tablets before swallowing since AMPYRA® is released slowly over time, and if the tablet is broken the medicine may be released too fast, which can raise the chance of having a seizure; if missing a dose of AMPYRA®, not to make up the missed dose; not to take 2 doses at the same time; to call the doctor or go to the nearest hospital emergency room right away if taking too much AMPYRA®; not to take AMPYRA® together with other aminopyridine medications, including compounded 4-AP (sometimes called 4-aminopyridine, fampridine) (The Ampyra® website)

The Acorda Website includes the following warnings relating to AMPYRA® administration: “AMPYRA® can cause seizures. Your chance of having a seizure is higher if you take too much AMPYRA® or if you have kidney problems. Before taking AMPYRA® tell your doctor if you have kidney problems. Stop taking AMPYRA® and call your doctor right away if you have a seizure while taking AMPYRA®; AMPYRA® may cause serious side effects, including kidney or bladder infections; The most common side effects of AMPYRA® include: urinary tract infection; trouble sleeping (insomnia); dizziness; headache; nausea; weakness; back pain; problems with balance; multiple sclerosis relapse; burning, tingling or itching of your skin; irritation in your nose and throat; constipation; indigestion; pain in your throat (The Acorda website).

AMPYRA® was approved by the US authorities as a symptomatic treatment aimed to improve walking ability in MS patients, which is central to the patient quality of life. As a symptomatic treatment, AMYPRA is expected to be used as an add-on drug in order to specifically improve the walking ability of patients which have reached a stage of ambulatory difficulties. However, a risk for serious side effects is associated with the currently recommended (optimal) AMYPRA dose and/or regimen.

### Laquinimod

Laquinimod is a novel synthetic compound with high oral bioavailability which has been suggested as an oral formulation for the treatment of Multiple Sclerosis (MS) (Polman, 2005; Sandberg-Wollheim, 2005; Comi et al 2008). Laquinimod and its sodium salt form are described, for example, in U.S. Pat. No. 6,077,851. The mechanism of action of laquinimod is not fully understood.

Animal studies show that laquinimod causes a Th1 (T helper 1 cell, produces pro-inflammatory cytokines) to Th2 (T helper 2 cell, produces anti-inflammatory cytokines) shift with an anti-inflammatory profile (Yang, 2004; Bruck, 2011). Another study demonstrated (mainly via the NFkB pathway) that laquinimod induced suppression of genes related to antigen presentation and corresponding inflammatory pathways (Gurevich, 2010). Other suggested potential mechanisms of action include inhibition of leukocyte migration into the CNS, increase of axonal integrity, modulation of cytokine production, and increase in levels of brain-derived neurotrophic factor (BDNF) (Runström, 2006; Bruck, 2011).

Laquinimod showed a favorable safety and tolerability profile in two phase III trials (Results of Phase III BRAVO Trial Reinforce Unique Profile of Laquinimod for Multiple Sclerosis Treatment; Teva Pharma, Active Biotech Post Positive Laquinimod Phase 3 ALLEGRO Results).

### Combination Therapy

The effects of combination therapy using laquinimod and fampridine on MS patients have not been reported.

The administration of two drugs to treat a given condition, such as multiple sclerosis, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry, 1999). In one example, combined administration of GA and interferon (IFN) has been experimentally shown to abrogate the clinical effectiveness of either therapy. (Brod 2000) In another experiment, it was reported that the addition of prednisone in combination therapy with IFN-β antagonized its up-regulator effect. Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry, 1999). The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects. (Vollmer, 2008; Rudick 2006; Kleinschmidt-DeMasters, 2005; Langer-Gould 2005)

Additionally, it is difficult to accurately predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry, 1999).

Therefore, the state of the art at the time of filing is that the effects of a combination therapy of two drugs, in particular laquinimod and fampridine, cannot be predicted until experimental results are available.

### SUMMARY OF THE INVENTION

The subject invention provides a method of treating a subject afflicted with multiple sclerosis or presenting a clinically isolated syndrome comprising periodically administering to the subject an amount of laquinimod, and an amount of fampridine, wherein the amounts when taken together are effective to treat the subject. In an embodiment, the amount of laquinimod and the amount of fampridine when administered together is more effective to treat the subject than when each agent at the same amount is administered alone.

The subject invention also provides a package comprising: a) a first pharmaceutical composition comprising an amount of laquinimod and a pharmaceutically acceptable carrier; b) a second pharmaceutical composition comprising an amount of fampridine and a pharmaceutically acceptable carrier; and c) instructions for use of the first and second pharmaceutical compositions together to treat a subject afflicted with multiple sclerosis or presenting a clinically isolated syndrome.

The subject invention also provides laquinimod for use as an add-on therapy or in combination with fampridine in treating a subject afflicted with multiple sclerosis or presenting a clinically isolated syndrome.

The subject invention also provides a pharmaceutical composition comprising an amount of laquinimod and an amount of fampridine for use in treating a subject afflicted with multiple sclerosis or presenting a clinically isolated syndrome, wherein the laquinimod and the fampridine are administered simultaneously or contemporaneously.

The subject invention also provides use of an amount of laquinimod and an amount of fampridine in the preparation of a combination for treating a subject afflicted with multiple sclerosis or presenting a clinically isolated syndrome wherein the laquinimod and the fampridine are administered simultaneously or contemporaneously.

The subject invention also provides a pharmaceutical composition comprising an amount of laquinimod for use in treating a subject afflicted with multiple sclerosis or presenting a clinically isolated syndrome in combination with fampridine by periodically administering the pharmaceutical composition and the fampridine to the subject.

The subject invention also provides a pharmaceutical composition comprising an amount of fampridine for use treating a subject afflicted with multiple sclerosis or presenting a clinically isolated syndrome in combination with laquinimod by periodically administering the pharmaceutical composition and the laquinimod to the subject.

### BRIEF DESCRIPTION OF THE DRAWINGS

FIG. 1 is a graphical representation of the experimental results from Example 1. The graph shows the clinical score for the EAE rodents in each group (on the y-axis) against the days after induction of the disease (on the x-axis).

### DETAILED DESCRIPTION OF THE INVENTION

The subject invention provides a method of treating a subject afflicted with multiple sclerosis or presenting a clinically isolated syndrome comprising periodically administering to the subject an amount of laquinimod, and an amount of fampridine, wherein the amounts when taken together are effective to treat the subject. In an embodiment, the amount of laquinimod and the amount of fampridine when administered together is more effective to treat the subject than when each agent at the same amount is administered alone.

In one embodiment, the laquinimod is laquinimod sodium. In another embodiment the fampridine is fampridine chloride.

In one embodiment, fampridine is administered in a slow release form. In another embodiment, the laquinimod and/or the fampridine is administered via oral administration. In another embodiment, the laquinimod and/or the fampridine is administered once daily. In yet another embodiment, the laquinimod and/or the fampridine is administered twice daily.

In one embodiment, the amount laquinimod administered is less than 0.6 mg/day. In another embodiment, the amount laquinimod administered is 0.25-2.0 mg/day. In another embodiment, the amount laquinimod administered is 0.25 mg/day. In another embodiment, the amount laquinimod administered is 0.3 mg/day. In another embodiment, the amount laquinimod administered is 0.5-1.2 mg/day. In another embodiment, the amount laquinimod administered is 0.5 mg/day. In another embodiment, the amount laquinimod administered is 0.6 mg/day. In another embodiment, the amount laquinimod administered is 1.0 mg/day. In yet another embodiment, the amount laquinimod administered is 1.2 mg/day.

In one embodiment, the amount fampridine administered is less than 20 mg/day. In another embodiment, the amount fampridine administered is 1.0-20 mg/day. In another embodiment, the amount fampridine administered is 2.5 mg/day. In another embodiment, the amount fampridine administered is 5-15 mg/day. In another embodiment, the amount fampridine administered is 5 mg/day. In another embodiment, the amount fampridine administered is 10 mg/day. In yet another embodiment, the amount fampridine administered is 15 mg/day.

In one embodiment, the amount fampridine administered is 1.25 mg b.i.d. In another embodiment, the amount fampridine administered is 2.5 mg b.i.d. In another embodiment, the amount fampridine administered is 5 mg b.i.d. In yet another embodiment, the amount fampridine administered is 7.5 mg b.i.d.

In an embodiment, the amount of laquinimod and the amount of fampridine when taken together is effective to alleviate a symptom of multiple sclerosis in the subject. In another embodiment, the symptom is a MRI-monitored multiple sclerosis disease activity, relapse rate, accumulation of physical disability, frequency of relapses, frequency of clinical exacerbation, brain atrophy, risk for confirmed progression, time to confirmed disease progression, visual function, fatigue or impaired mobility.

In one embodiment, the accumulation of physical disability is measured by the subject's Kurtzke Expanded Disability Status Scale (EDSS) score. In another embodiment, the accumulation of physical disability is assessed by the time to confirmed disease progression as measured by Kurtzke Expanded Disability Status Scale (EDSS) score.

In one embodiment, the impaired mobility is assessed by the Timed-25 Foot Walk test. In another embodiment, the impaired mobility is assessed by the MSWS-12 self-report questionnaire. In another embodiment, the impaired mobility is assessed by the Ambulation Index. In another embodiment, the mobility is assessed by the Six-Minute Walk (6MW) Test. In another embodiment, the impaired mobility is assessed by the LEMMT Test.

In one embodiment, the amount of laquinimod and the amount of fampridine when taken together is effective to improve the subject's mobility. In another embodiment, the amount of laquinimod and the amount of fampridine when taken together is effective to improve the subject's quality of life. In another embodiment, the quality of life is assessed by the SF-36 Test, EQ-5D, Subject Global Impression (SGI) or Clinician Global Impression of Change (CGIC). In another embodiment, the amount of laquinimod and the amount of fampridine when taken together is effective to improve the general health status of the subject. In another embodiment, the general health status is assessed by the EQ-5D, Subject Global Impression (SGI) or Clinician Global Impression of Change (CGIC). In another embodiment, the fatigue is assessed by the EQ-5D or the EMIF-SEP score.

In one embodiment, the administration of laquinimod substantially precedes the administration of fampridine. In another embodiment, the administration of fampridine substantially precedes the administration of laquinimod. In yet another embodiment, the subject is receiving laquinimod therapy prior to initiating fampridine therapy.

In one embodiment of the present invention, the method further comprises administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies. In another embodiment of the present invention, the administration of laquinimod and fampridine inhibits a symptom of relapsing multiple sclerosis by at least 30%.

In one embodiment, each of the amount of laquinimod when taken alone, and the amount of fampridine when taken alone is effective to treat the subject. In another embodiment, either the amount of laquinimod or when taken alone, the amount of fampridine when taken alone, or each such amount when taken alone is not effective to treat the subject. In yet another embodiment, the subject is a human.

The subject invention also provides a package comprising: a) a first pharmaceutical composition comprising an amount of laquinimod and a pharmaceutically acceptable carrier; b) a second pharmaceutical composition comprising an amount of fampridine and a pharmaceutically acceptable carrier; and c) instructions for use of the first and second pharmaceutical compositions together to treat a subject afflicted with multiple sclerosis or presenting a clinically isolated syndrome.

The subject invention also provides laquinimod for use as an add-on therapy or in combination with fampridine in treating a subject afflicted with multiple sclerosis or presenting a clinically isolated syndrome.

The subject invention also provides a pharmaceutical composition comprising an amount of laquinimod and an amount of fampridine for use in treating a subject afflicted with multiple sclerosis or presenting a clinically isolated syndrome, wherein the laquinimod and the fampridine are administered simultaneously or contemporaneously.

In one embodiment the laquinimod is laquinimod sodium. In another embodiment, the fampridine is fampridine chloride.

In one embodiment, the pharmaceutical composition is for use in improving the subject's mobility.

In an embodiment, the amount of laquinimod in the composition is less than 0.6 mg. In another embodiment, the amount of laquinimod in the composition is 0.25-2.0 mg. In another embodiment, the amount of laquinimod in the composition is 0.25 mg. In another embodiment, the amount of laquinimod in the composition is 0.3 mg. In another embodiment, the amount of laquinimod in the composition is 0.5-1.2 mg. In another embodiment, the amount of laquinimod in the composition is 0.5 mg. In another embodiment, the amount of laquinimod in the composition is 0.6 mg. In another embodiment, the amount of laquinimod in the composition is 1.0 mg. In yet another embodiment, the amount of laquinimod in the composition is 1.2 mg.

In one embodiment, the amount of fampridine in the composition is less than 20 mg. In another embodiment, the amount of fampridine in the composition is 1.0-20 mg. In another embodiment, the amount of fampridine in the composition is 2.5 mg. In another embodiment, the amount of fampridine in the composition is 5-15 mg. In another embodiment, the amount of fampridine in the composition is 5 mg. In another embodiment, the amount of fampridine in the composition is 10 mg. In yet another embodiment, the amount of fampridine in the composition is 15 mg.

The subject invention also provides use of an amount of laquinimod and an amount of fampridine in the preparation of a combination for treating a subject afflicted with multiple sclerosis or presenting a clinically isolated syndrome wherein the laquinimod and the fampridine are administered simultaneously or contemporaneously.

In an embodiment, for the package, laquinimod, the pharmaceutical composition, or the use described herein, the multiple sclerosis is relapsing multiple sclerosis. In another embodiment, the relapsing multiple sclerosis is relapsing-remitting multiple sclerosis.

The subject invention also provides a pharmaceutical composition comprising an amount of laquinimod for use in treating a subject afflicted with multiple sclerosis or presenting a clinically isolated syndrome in combination with fampridine by periodically administering the pharmaceutical composition and the fampridine to the subject.

The subject invention also provides a pharmaceutical composition comprising an amount of fampridine for use treating a subject afflicted with multiple sclerosis or presenting a clinically isolated syndrome in combination with laquinimod by periodically administering the pharmaceutical composition and the laquinimod to the subject.

For the foregoing embodiments, each embodiment disclosed herein is contemplated as being applicable to each of the other disclosed embodiment.

A pharmaceutically acceptable salt of laquinimod as used in this application includes lithium, sodium, potassium, magnesium, calcium, manganese, copper, zinc, aluminum and iron. Salt formulations of laquinimod and the process for preparing the same are described, e.g., in U.S. Pat. No. 7,589,208 and PCT International Application Publication No. WO 2005/074899, which are hereby incorporated by reference into this application.

Laquinimod can be administered in admixture with suitable pharmaceutical diluents, extenders, excipients, or carriers (collectively referred to herein as pharmaceutically acceptable carriers) suitably selected with respect to the intended form of administration and as consistent with conventional pharmaceutical practices. The unit will be in a form suitable for oral administration. Laquinimod can be administered alone but is generally mixed with a pharmaceutically acceptable carrier, and co-administered in the form of a tablet or capsule, liposome, or as an agglomerated powder. Examples of suitable solid carriers include lactose, sucrose, gelatin and agar.

Capsule or tablets can be formulated and can be made easy to swallow or chew; other solid forms include granules, and bulk powders.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, dicalcium phosphate, calcium sulfate, mannitol, sorbitol, microcrystalline cellulose and the like. Suitable binders include starch, gelatin, natural sugars such as glucose or beta-lactose, corn starch, natural and synthetic gums such as acacia, tragacanth, or sodium alginate, povidone, carboxymethylcellulose, polyethylene glycol, waxes, and the like. Lubricants used in these dosage forms include sodium oleate, sodium stearate, sodium benzoate, sodium acetate, sodium chloride, stearic acid, sodium stearyl fumarate, talc and the like. Disintegrators (disintegrants) include, without limitation, starch, methyl cellulose, agar, bentonite, xanthan gum, croscarmellose sodium, sodium starch glycolate and the like.

Specific examples of the techniques, pharmaceutically acceptable carriers and excipients that may be used to formulate oral dosage forms of the present invention are described, e.g., in U.S. Pat. No. 7,589,208, PCT International Application Publication Nos. WO 2005/074899, WO 2007/047863, and 2007/146248.

General techniques and compositions for making dosage forms useful in the present invention are described-in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds). These references in their entireties are hereby incorporated by reference into this application.

Disclosed is a method for treating a subject, e.g., human patient, afflicted with relapsing multiple sclerosis using a combination of laquinimod and fampridine. The use of laquinimod for relapsing multiple sclerosis had been previously suggested in, e.g., U.S. Pat. No. 6,077,851 and the symptomatic use of fampridine to treat walking deficits in patients with multiple sclerosis has been approved by the FDA (FDA News Release, The Acorda Website). However, the use of fampridine in its recommended dose and regimen is associated with considerable risks for serious side effects. The inventors have surprisingly found that the combination of laquinimod and fampridine at a lowered dose is more effective for the treatment of relapsing multiple sclerosis, and specifically in the improvement of mobility, than an optimal dose of fampridine alone.

### TERMS

As used herein, and unless stated otherwise, each of the following terms shall have the definition set forth below.

As used herein, “laquinimod” means laquinimod acid or a pharmaceutically acceptable salt thereof.

As used herein, “fampridine” means fampridine or a pharmaceutically acceptable salt thereof.

As used herein, an “amount” or “dose” of laquinimod as measured in milligrams refers to the milligrams of laquinimod acid present in a preparation, regardless of the form of the preparation. A “dose of 0.6 mg laquinimod” means the amount of laquinimod acid in a preparation is 0.6 mg, regardless of the form of the preparation. Thus, when in the form of a salt, e.g. a laquinimod sodium salt, the weight of the salt form necessary to provide a dose of 0.6 mg laquinimod would be greater than 0.6 mg (e.g., 0.64 mg) due to the presence of the additional salt ion. Similarly, the “amount” or “dose” of fampridine as measured in milligrams refers to the milligrams of fampridine present in a preparation, regardless of the form of preparation. Thus, when in the form of a salt, e.g. fampridine chloride, the weight of the salt form necessary to provide a dose of 10 mg laquinimod would be greater than 10 mg due to the presence of the additional salt ion.

It is understood that when an amount administered daily is provided, this specific amount is the total amount given over a period of 24 hours. For example, fampridine administered 20 mg daily or 20 mg/day means the total amount of fampridine administered over a 24 hours period is 20 mg. The amount of 20 mg may be administered once daily, in two doses of 10 mg each, in four doses of 5 mg each, and so on. An administration of a certain amount b.i.d refers to two doses of the amount over a 24-hour period. For example, 10 mg b.i.d refers to two doses of 10 mg each given over a 24-hour period.

As used herein, “about” in the context of a numerical value or range means ±10% of the numerical value or range recited or claimed.

As used herein, “combination” means an assemblage of reagents for use in therapy either by simultaneous or contemporaneous administration. Simultaneous administration refers to administration of an admixture (whether a true mixture, a suspension, an emulsion or other physical combination) of the laquinimod and the fampridine. In this case, the combination may be the admixture or separate containers of the laquinimod and the fampridine that are combined just prior to administration. Contemporaneous administration refers to the separate administration of the laquinimod and the fampridine at the same time, or at times sufficiently close together that a synergistic activity relative to the activity of either the laquinimod or the fampridine alone is observed.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding fampridine therapy to a patient already receiving laquinimod therapy.

“Administering to the subject” means the giving of, dispensing of, or application of medicines, drugs, or remedies to a subject to relieve, cure, or reduce the symptoms associated with a condition, e.g., a pathological condition.

As used herein, “effective” when referring to an amount of laquinimod and/or fampridine refers to the quantity of laquinimod and/or fampridine that is sufficient to yield a desired therapeutic response without undue adverse side effects (such as toxicity, irritation, or allergic response) commensurate with a reasonable benefit/risk ratio when used in the manner of this invention.

“Treating” as used herein encompasses, e.g., inducing inhibition, regression, or stasis of a disease or disorder, e.g., RMS, or alleviating, lessening, suppressing, inhibiting, reducing the severity of, eliminating or substantially eliminating, or ameliorating a symptom of the disease or disorder. “Treating” as applied to patients presenting CIS can mean delaying the onset of clinically definite multiple sclerosis (CDMS), delaying the progression to CDMS, reducing the risk of conversion to CDMS, or reducing the frequency of relapse in a patient who experienced a first clinical episode consistent with multiple sclerosis and who has a high risk of developing CDMS.

“Inhibition” of disease progression or disease complication in a subject means preventing or reducing the disease progression and/or disease complication in the subject.

A “symptom” associated with multiple sclerosis includes any clinical or laboratory manifestation associated with RMS and is not limited to what the subject can feel or observe.

As used herein, “a subject afflicted with” a disease means a subject who was affirmatively diagnosed to have the disease. For example, “a subject afflicted with relapsing multiple sclerosis” means a subject who was been affirmatively diagnosed to have relapsing multiple sclerosis (RMS) which includes relapsing-remitting multiple sclerosis (RRMS) and Secondary Progressive multiple sclerosis (SPMS).

“Relapse Rate” is the number of confirmed relapses per unit time. “Annualized relapse rate” is the mean value of the number of confirmed relapses of each patient multiplied by 365 and divided by the number of days that patient is on the study drug.

“Expanded Disability Status Scale” or “EDSS” is a rating system that is frequently used for classifying and standardizing the condition of people with multiple sclerosis. The score ranges from 0.0 representing a normal neurological exam to 10.0 representing death due to MS. The score is based upon neurological testing and examination of functional systems (FS), which are areas of the central nervous system which control bodily functions. The functional systems are: Pyramidal (ability to walk), Cerebellar (coordination), Brain stem (speech and swallowing), Sensory (touch and pain), Bowel and bladder functions, Visual, Mental, and Other (includes any other neurological findings due to MS) (Kurtzke J F, 1983).

The “Six-Minute Walk (6MW) Test” is a commonly used test developed to assess exercise capacity in patients with COPD (Guyatt, 1985). It has been used also to measure mobility in multiple sclerosis patients (Clinical Trials Website).

A “confirmed progression” of EDSS, or “confirmed disease progression” as measured by EDSS score is defined as a 1 point increase from baseline EDSS if baseline EDSS was between 0 and 5.0, or a 0.5 point increase if baseline EDSS was 5.5 or more. In order to be considered a confirmed progression, the change (either 1 point or 0.5 points) must be sustained for at least 3 months. In addition, confirmation of progression cannot be made during a relapse.

“Adverse event” or “AE” means any untoward medical occurrence in a clinical trial subject administered a medicinal product and which does not have a causal relationship with the treatment. An adverse event can therefore be any unfavorable and unintended sign including an abnormal laboratory finding, symptom, or diseases temporally associated with the use of an investigational medicinal product, whether or not considered related to the investigational medicinal product.

“Gd-enhancing lesion” refers to lesions that result from a breakdown of the blood-brain barrier, which appear in contrast studies using gadolinium contrast agents. Gadolinium enhancement provides information as to the age of a lesion, as Gd-enhancing lesions typically occur within a six week period of lesion formation.

“Magnetization Transfer Imaging” or “MTI” is based on the magnetization interaction (through dipolar and/or chemical exchange) between bulk water protons and macromolecular protons. By applying an off resonance radio frequency pulse to the macromolecular protons, the saturation of these protons is then transferred to the bulk water protons. The result is a decrease in signal (the net magnetization of visible protons is reduced), depending on the magnitude of MT between tissue macromolecules and bulk water. “MT” or “Magnetization Transfer” refers to the transfer of longitudinal magnetization from the hydrogen nuclei of water that have restricted motion to the hydrogen nuclei of water that moves with many degrees of freedom. With MTI, the presence or absence of macromolecules (e.g. in membranes or brain tissue) can be seen (Mehta, 1996; Grossman, 1994).

“Magnetization Resonance Spectroscopy” or “MRS” is a specialized technique associated with magnetic resonance imaging (MRI). MRS is used to measure the levels of different metabolites in body tissues. The MR signal produces a spectrum of resonances that correspond to different molecular arrangements of the isotope being “excited”. This signature is used to diagnose certain metabolic disorders, especially those affecting the brain, (Rosen, 2007) as well as to provide information on tumor metabolism (Golder, 2007).

As used herein “mobility” refers to any ability relating to walking, walking speed, gait, strength of leg muscles, leg function and the ability to move with or without assistance. Mobility can be evaluated by one or more of several tests including but not limited to Ambulation Index, Time 25 foot walk, Six-Minute Walk (6MW), Lower Extremity Manual Muscle Test (LEMMT), and EDSS. Mobility can also be reported by the subject, for example by questionnaires, including but not limited to 12-Item Multiple Sclerosis Walking Scale (MSWS-12). Impaired Mobility refers to any impairment, difficulty or disability relating to mobility.

“T1-weighted MRI image” refers to an MR-image that emphasizes T1 contrast by which lesions may be visualized. Abnormal areas in a T1-weighted MRI image are “hypointense” and appear as dark spots. These spots are generally older lesions.

“T2-weighted MRI image” refers to an MR-image that emphasizes T2 contrast by which lesions may be visualized. T2 lesions represent new inflammatory activity.

A “patient at risk of developing MS” (i.e. clinically definite MS) as used herein is a patient presenting any of the known risk factors for MS. The known risk factors for MS include any one of clinically isolated syndrome (CIS), a single attack suggestive of MS without a lesion, the presence of a lesion (in any of the CNS, PNS, or myelin sheath) without a clinical attack, environmental factors (geographical location, climate, diet, toxins, sunlight), genetics (variation of genes encoding HLA-DRB1, IL7R-alpha and IL2R-alpha), and immunological components (viral infection such as by Epstein-Barr virus, high avidity CD4⁺ T cells, CD8⁺ T cells, anti-NF-L, anti-CSF 114(Glc)).

“Clinically isolated syndrome (CIS)” as used herein refers to 1) a single clinical attack (used interchangeably herein with “first clinical event” and “first demyelinating event”) suggestive of MS, which, for example, presents as an episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning, and 2) at least one lesion suggestive of MS. In a specific example, CIS diagnosis would be based on a single clinical attack and at least 2 lesions suggestive of MS measuring 6 mm or more in diameter.

A “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

The “Timed-25 Foot Walk” or “T25-FW” is a quantitative mobility and leg function performance test based on a timed 25-walk. The patient is directed to one end of a clearly marked 25-foot course and is instructed to walk 25 feet as quickly as possible, but safely. The time is calculated from the initiation of the instruction to start and ends when the patient has reached the 25-foot mark. The task is immediately administered again by having the patient walk back the same distance. Patients may use assistive devices when doing this task. The score for the T25-FW is the average of the two completed trials. This score can be used individually or used as part of the MSFC composite score (National MS Society Website).

One of the central symptoms of multiple sclerosis is fatigue. Fatigue can be measured by several tests including but not limited to decrease of EMIF-SEP score, and EQ-5D. Other tests, including but not limited to Clinician Global Impression of Change (CGIC) and Subject Global Impression (SGI), as well as EQ-5D, can be used to evaluate the general health status and quality of life of MS patients.

“Ambulation Index” or “AI” is a rating scale developed by Hauser et al. to assess mobility by evaluating the time and degree of assistance required to walk 25 feet. Scores range from 0 (asymptomatic and fully active) to 10 (bedridden). The patient is asked to walk a marked 25-foot course as quickly and safely as possible. The examiner records the time and type of assistance (e.g., cane, walker, crutches) needed. (Hauser, 1983)

“EQ-5D” is a standardized questionnaire instrument for use as a measure of health outcome applicable to a range of health conditions and treatments. It provides a simple descriptive profile and a single index value for health status that can be used in the clinical and economic evaluation of health care as well as population health surveys. EQ-5D was developed by the “EuroQoL” Group which comprises a network of international, multilingual, multidisciplinary researchers, originally from seven centers in England, Finland, the Netherlands, Norway and Sweden. The EQ-5D questionnaire is in the public domain and can be obtained from EuroQoL.

“SF-36” is a multi-purpose, short-form health survey with 36 questions which yields an 8-scale profile of functional health and well-being scores as well as psychometrically-based physical and mental health summary measures and a preference-based health utility index. It is a generic measure, as opposed to one that targets a specific age, disease, or treatment group. The survey is developed by and can be obtained from QualityMetric, Inc. of Providence, R.I.

It is understood that where a parameter range is provided, all integers within that range, and hundredth thereof, are also provided by the invention. For example, “0.25-2.0 mg/day” includes 0.25 mg/day, 0.26 mg/day, 0.27 mg/day, etc. up to 2.0 mg/day.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### Assessment of Efficacy of Laquinimod Alone or in-Combination with Fampridine in MOG-Induced EAE

In this experiment, MOG-induced EAE Mice were treated with a sub-optimal dose of laquinimod (10 mg/kg) alone or in combination with fampridine (2.5 mg/kg) to assess the efficacy of laquinimod alone or in combination with fampridine. MOG-induced Experimental Autoimmune Encephalomyelitis (EAE) in the C57B1 strain of mice is an established EAE model to test the efficacy of candidate molecules for MS treatment.

The dosages were chosen based on known effective dose amounts for laquinimod (0.6 mg/day) and for fampridine (10 mg/b.i.d) in humans (U.S. Patent Application Publication 2010-0322900; United Spinal's MS Scene). The National Institutes of Health (NIH) provides a table of Equivalent Surface Area Dosage Conversion Factors below (Table 1) which provides conversion factors that account for surface area to weight ratios between species.

TABLE 1
Equivalent Surface Area Dosage Conversion Factors

| | To | | | | | |
|---|---|---|---|---|---|---|
| | | Mouse | | Monkey | | |
| | | 20 g | Rat 150 g | 3 kg | Dog 8 kg | Man 60 kg |
| FROM | Mouse | 1 | ½ | ¼ | ⅙ | 1/12 |
| | Rat | 2 | 1 | ½ | ¼ | 1/7 |
| | Monkey | 4 | 2 | 1 | ⅗ | ⅓ |
| | Dog | 6 | 4 | 1⅔ | 1 | ½ |
| | Man | 12 | 7 | 3 | 2 | 1 |

Therefore, given a dose of 0.6 mg laquinimod in a human man, assuming body weight at 60 kg, the dose per kg would be 0.6 mg*60 kg⁻¹=0.01 mg/kg in a human. The corresponding dosage in the mouse is approximately 0.01 mg/kg*12=0.12 mg/kg. Similarly, given a dose of 20 mg/day fampridine in a human, the corresponding dosage in a mouse is approximately 4 mg/kg. The amounts used in this study were additionally adjusted based on previous work with the model used to the amounts herein.

Accordingly, the data from this mice study is representative of what can be expected in human patients with the treatment of laquinimod and fampridine at the corresponding human dosages.

### Procedure

Disease was induced in all mice by the injection of the encephalitogenic emulsion (MOG/CFA) and intraperitoneal injection of pertussis toxin on the first day and 48 hours later.
Fampridine at dose levels of 2.5 (sub optimal) and 5 mg/kg (optimal) were administered by the oral dose, once daily (QD).
Laquinimod at dose levels of 10 (sub optimal) and 25 mg/kg (optimal) were administered by the oral route, once daily (QD).
Both fampridine and laquinimod administration was initiated on the day of induction. Both fampridine and laquinimod were given by oral gavage daily with at least 4-hour interval, for a period of 30 days.

### Induction of EAE:

EAE was induced by subcutaneous injection of encephalitogenic emulsion at a volume of 0.2 ml/mouse into the flanks of the mice at two injection sites. On the day of induction, pertussis toxin was injected i.p. at a volume dose of 0.2 ml/mouse. The injection of the pertussis toxin was repeated after 48 hours.

### Test Procedure:

Day 0: Subcutaneous injection of MOG into right flank, i.p. injection of Pertussis toxin, beginning of daily laquinimod and fampridine treatment.

Day 2: i.p. injection of Pertussis toxin.

Day 10: Initiation of scoring of mice for EAE clinical signs.

Day 30: Termination of study.

### Materials:

1. Fampridine
2. Laquinimod
3. Mycobacterium tuberculosis (MT), Difco
4. Pertussis toxin, Sigma
5. MOG 35-55, Mnf Novatide
6. Complete Freund's Adjuvant (CFA), Sigma
7. Saline, Mnf—DEMO S.A
8. Sterile double distilled water (DDW)

### Experimental Animals:

Healthy, nulliparous, non-pregnant female mice of the C57BL/6 strain were used in the study. The animals weighed 18-22 grams, and were approximately 8 weeks old on receipt.

The body weights of the animals were recorded on the day of delivery. Overtly healthy animals were assigned to study groups arbitrarily before treatment commenced.

The mice were individually identified by using ear tags. A color-coded card on each cage gave information including cage number, group number and identification.

### EAE Induction:

EAE was induced by injecting the encephalitogenic mixture (emulsion) consisting of MOG (150.0 μg/mouse) and CFA containing M. tuberculosis (2 mg MT/mL CFA).

A volume of 0.2 ml of emulsion was injected subcutaneously into the flanks of the mice.

Pertussis toxin in 0.2 ml dosage volume was injected intraperitoneally on the day of induction and 48 hours later (total amount is 0.1+0.1=0.2 μg/mouse).

### Study Design:

The mice were allocated randomly into 7 groups according to Table 2 below.

| | Treatment | | Administration |
|---|---|---|---|
| Group | Groups | dose/day | Route |
| 1 | DDW | | oral |
| | (negative control) | | |
| 2 | Laquinimod | 10 mg/kg | oral |
| 3 | | 25 mg/kg | oral |
| 4 | 4-aminopyridine | 2.5 mg/kg | oral |
| 5 | (4AP) | 5 mg/kg | oral |
| 6 | Laquinimod + 4AP | 10 mg/kg + 2.5 mg/kg | oral |
| 7 | Laquinimod + 4AP | 10 mg/kg + 5 mg/kg | oral |

### Preparation and Administration of Encephalitogenic Emulsion:

Oil portion: 32.1 ml CFA (containing 2 mg/ml MT).

Liquid portion: 48.2 MOG or equivalent was diluted in 32.1 ml Normal Saline to yield 1.5 mg/ml MOG stock solution.

The emulsion was made from equal parts of oil and liquid portions (1:1) in two syringes connected to each other with Leur lock to yield 0.75 mg/ml and 1 mg/ml MT. The emulsion was transferred to insulin syringe and 0.2 ml was injected to the right flank of each mouse. Dose=0.15 mg MOG and 0.2 mg MT/mouse.

### Preparation and Administration of Pertussis Toxin:

75 μL Pertussis toxin (200 μg/ml) was added to 29.925 ml saline to yield 500 ng/ml. The pertussis toxin was administered intraperitoneally on the day of encephalitogen injection and 48 hours later (100.0 ng/0.2 ml/mouse)—Total 200 ng/mouse.

### Preparation and Administration of Test Articles

### Fampridine Formulations:

Fampridine was weighed and sterile DDW was added to yield 0.25 and 0.5 mg/ml for dose levels of 2.5 and 5.0 mg/kg respectively. The mice were administered with the two concentrations of fampridine (0.25 and 0.5 mg/ml), a volume dose level of 200 μl/mouse by the oral route for dose levels of 2.5 and 5.0 mg/kg respectively.

### Laquinimod Formulations:

A concentration of 1.0 and 2.5 mg/ml laquinimod was prepared in DDW. The test formulations were stored at 2-8° C. until use in amber colored bottles.

The mice were administered with the two concentrations of laquinimod (1.0 and 2.5 mg/ml), a volume dose level of 200 μl/mouse by the oral route for dose levels of 10 and 25 mg/kg respectively.

Both the fampridine and the laquinimod formulations were administered from Day 1, once daily (QD).

Four hours interval was maintained daily between administration of laquinimod and fampridine.

### EAE Clinical Signs:

The mice were observed daily from the 10th day post-EAE induction (first injection of MOG) and the EAE clinical signs were scored according to the grades described in the table presented below.

TABLE 3
Evaluation of the EAE clinical signs

| Score | Signs | Description |
|---|---|---|
| 0 | Normal behavior | No neurological signs. |
| 1 | Limp tail | Part or the whole tail is limp and droopy. |
| 2 | Righting reflex | Animal has difficulties rolling onto his feet |
| | | when laid on its back |
| 3 | Hind leg | Wobbly walk - when the mouse walks the hind |
| | | legs are unsteady weakness |
| 4 | Hind leg paralysis | The mouse drags its hind legs but is able to |
| | | move around using its fore legs |
| 5 | Full paralysis | The mouse can't move around, it looks thinner |
| | | and emaciated. |
| 6 | Moribund/Death | |

All mice with score 1 and above were considered sick. Animals with score 4 for more than two days were given score 5 and sacrificed for humane reasons. For calculation purposes, the score (5) of animals that were sacrificed or died was carried forward (LOCF).

### Acceptance Criteria

Acceptance criteria for Negative Control Group:

The control group should have at least 70% incidence.

The MMS should be more than 2.0.

### Interpretation of Results

### Calculation of the Incidence of Disease (Disease Ratio)

The number of sick animals in each group were summed.
   The incidence of disease was calculated as

$$INCIDENCE of DISEASE = ( \frac{No . of sick mice in treated group}{No . of sick mice in control group} )$$

The percent inhibition according to incidence was calculated as

$$INHIBITION ( \% ) of INCIDENCE = ( 1 - \frac{Number of sick mice in treated group}{Number of sick mice in control group} ) \times 100$$

### Calculation of the Mortality/Moribundity Rate (Mortality Ratio)

The number of dead or moribund animals in each group were summed.
The mortality of disease was calculated as

$$MORTALITY of DISEASE = ( \frac{No . of dead or moribound mice in treated group}{No . of dead or moribound mice in control group} )$$

The percent inhibition according to mortality was calculated as

$$INHIBITION ( \% ) of MORTALITY = ( 1 - \frac{Number of dead or moribound mice in treated group}{Number of dead or moribound mice in control group} ) \times 100$$

### Calculation of Duration of Disease

The mean duration of disease expressed in days was calculated as

$$Mean Duration = ( \frac{\sum Duration of disease of each mouse}{No . of mice in th e group} )$$

### Calculation of Mean Delay in Onset of Disease

The mean onset of disease expressed in days was calculated as

$$Mean Onset = ( \frac{\sum Onset of disease of each mouse}{No . of mice in the group} )$$

The mean delay in onset of disease expressed in days was calculated by subtracting the mean onset of disease in control group from test group.

### Calculation of the Mean Maximal Score and Percent Inhibition

The mean maximal score (MMS) of each group was calculated as

$$MMS = ( \frac{\sum Maximal Score of each mouse}{No . of mice in the group} )$$

The percent inhibition according to MMS was calculated as

$$INHIBITION ( \% ) of MMS = ( 1 - \frac{MMS of treated group}{MMS of control group} ) \times 100$$

### Calculation of the Group Mean Score and Percent Inhibition

The daily scores of each mouse in the test group were summed and the individual mean daily score (IMS) was calculated as

$$IMS = ( \frac{\sum Daily score of mouse}{Observation period ( days )} )$$

The mean group score (GMS) was calculated as

$$GMS = ( \frac{\sum IMS of each mouse}{No . of mice in the group} )$$

The percent inhibition was calculated as

$$INHIBITION ( \% ) of GMS = ( 1 - \frac{GMS of treated group}{GMS of control group} ) \times 100$$

### Results

A summary of the incidence, mortality, Group Mean Score (GMS), duration of the disease, onset of the disease and the activity of each group compared to the vehicle treated control group is shown in the Summarized Table 4:

TABLE 4
Summary table - Mortality, incidence, MMS, GMS and duration
of EAE activity compared to vehicle

| | | | | | | | | Mean | Mean |
|---|---|---|---|---|---|---|---|---|---|
| | | | % | MMS | % | GMS | % | Onset | Onset |
| Treatment | Mortality | Incidence | inhibition 1 | value | inhibition 2 | value | inhibition 3 | (days) | (days) |
| DDW | 0/15 | 15/15 | — | 3.9 ± 0.4 | — | 2.9 ± 0.7 | — | 9.9 ± 1.4 | 21.1 ± 1.4 |
| (negative | | | | | | | | | |
| control) | | | | | | | | | |
| LAQ | 0/15 | 10/15 | 33.3% | 2.4 ± 1.9 | 38.5% | 1.2 ± 1.0 | 58.6% | 20.7 ± 7.9 | 10.3 ± 7.9 |
| (10 mg/kg) | | | | | (p = 0.07) | | (p < 0.001) | (p < 0.001) | (p < 0.001) |
| LAQ | 0/15 | 4/15 | 73.3% | 0.7 ± 1.3 | 82.1% | 0.4 ± 0.7 | 86.2% | 26.7 ± 7.5 | 4.3 ± 7.5 |
| (25 mg/kg) | | | | | (p < 0.001) | | (p < 0.001) | (p < 0.001) | (p < 0.001) |
| 4AP | 0/15 | 15/15 | 0% | 3.5 ± 0.6 | 10.2% | 2.2 ± 0.5 | 24.1% | 10.8 ± 1.0 | 20.2 ± 1.0 |
| (2.5 mg/kg) | | | | | (p = 0.2) | | (p = 0.051) | (p = 0.04) | (p = 0.04) |
| 4AP | 0/15 | 15/15 | 0% | 3.4 ± 0.6 | 12.8% | 1.9 ± 0.6 | 34.5% | 12.0 ± 1.1 | 19.0 ± 1.1 |
| (5 mg/kg) | | | | | (p = 0.6) | | (p = <0.001) | (p < 0.001) | (p < 0.001) |
| LAQ + 4AP | 0/15 | 9/15 | 40% | 1.7 ± 1.7 | 56.4% | 0.8 ± 0.8 | 72.4% | 21.7 ± 8.6 | 9.3 ± 8.6 |
| (10 mg + | | | | | (p < 0.001) | | (p < 0.001) | (p < 0.001) | (p < 0.001) |
| 2.5 mg/kg) | | | | | | | | | |
| LAQ + 4AP | 0/15 | 8/15 | 46.6% | 1.1 ± 1.3 | 71.8% | 0.5 ± 0.5 | 82.8% | 23.8 ± 7.6 | 7.2 ± 7.6 |
| (10 mg + | | | | | (p < 0.001) | | (p < 0.001) | (p < 0.001) | (p < 0.001) |
| 5 mg/kg) | | | | | | | | | |

A summary of the incidence, mortality, Group Mean Score (GMS), duration of the disease, onset of the disease and the activity of each group compared to laquinimod suboptimal dose is shown in the Summarized Table 5:

TABLE 5
Test formulations: Laquinimod (LAQ) alone and in
combination with Fampridine (4AP) EAE activity compared to LAQ (10 mg/kg)

| | | | | | | | | Mean | Mean |
|---|---|---|---|---|---|---|---|---|---|
| | | | % | MMS | % | GMS | % | Onset | Onset |
| Treatment | Mortality | Incidence | inhibition 1 | value | inhibition 2 | value | inhibition 3 | (days) | (days) |
| LAQ | 0/15 | 10/15 | — | 2.4 ± 1.9 | — | 1.2 ± 1.0 | — | 20.7 ± 7.9 | 10.3 ± 7.9 |
| (10 mg/kg) | | | | | | | | | |
| LAQ | 0/15 | 4/15 | 60.0% | 0.7 ± 1.3 | 70.8% | 0.4 ± 0.7 | 66.7% | 26.7 ± 7.5 | 4.3 ± 7.5 |
| (25 mg/kg) | | | | | (p = 0.021) | | (p = 0.037) | (p = 0.081) | (p = 0.081) |
| LAQ + 4AP | 0/15 | 9/15 | 40% | 1.7 ± 1.7 | 29.2% | 0.8 ± 0.8 | 33.3% | 21.7 ± 8.6 | 9.3 ± 8.6 |
| (10 mg + | | | | | (p = 0.25) | | (p = 0.23) | (p = 0.90) | p = 0.90) |
| 2.5 mg/kg) | | | | | | | | | |
| LAQ + 4AP | 0/15 | 8/15 | 46.6% | 1.1 ± 1.3 | 54.2% | 0.5 ± 0.5 | 58.3% | 23.8 ± 7.6 | 7.2 ± 7.6 |
| (10 mg + | | | | | (p = 0.056) | | (p = 0.074) | (p = 0.29) | (p = 0.29) |
| 5 mg/kg) | | | | | | | | | |

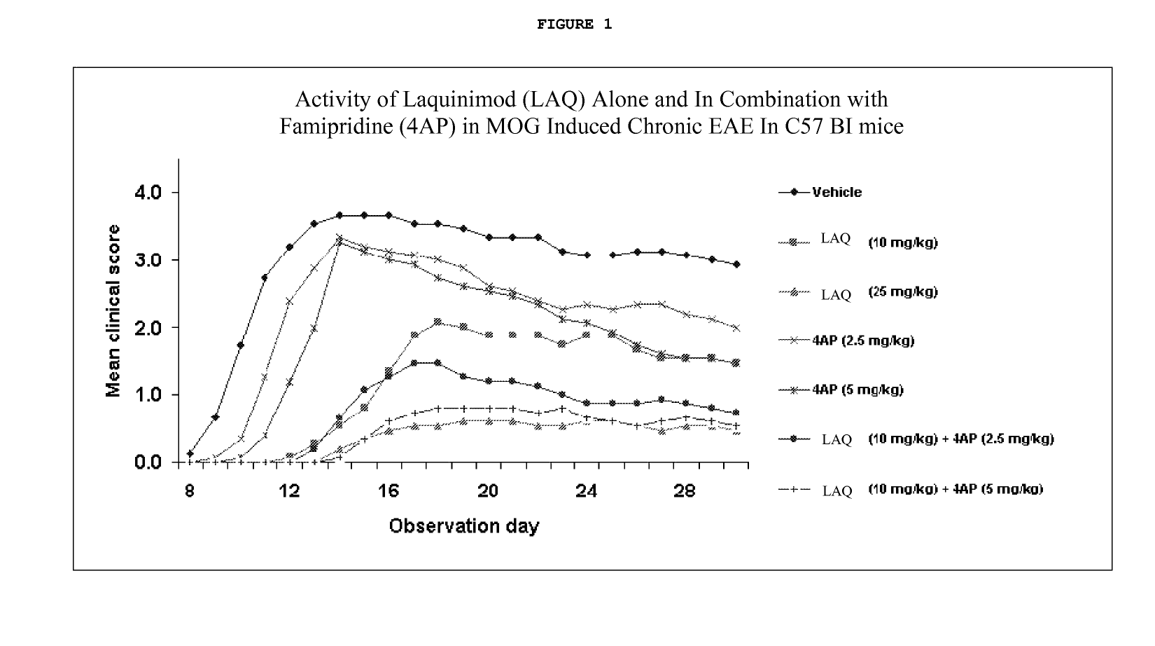

The Clinical profile of the treatment groups are presented graphically in FIG. 1.

In groups treated with fampridine at dose levels of 2.5 mg/kg (sub optimal dose), and 5.0 mg/kg (optimal dose), 24.1 and 34.5% activity, respectively, was observed according to GMS when compared to the vehicle administered control group.

In groups treated with laquinimod at dose levels of 10 mg/kg (sub optimal dose), and 25 mg/kg (optimal dose), 58.6 and 86.2% activity, respectively, was observed according to GMS when compared to the vehicle administered control group.

The combination of fampridine at sub optimal dose (2.5 mg/kg) with a sub optimal dose of laquinimod (10 mg/kg)-72.4% inhibition compared to vehicle, exhibited activity superior to the sub optimal dose of fampridine alone (2.5 mg/kg; 24.1% compared to vehicle) and laquinimod alone (10 mg/kg; 58.6% compared to vehicle).

Surprisingly, the combination of fampridine at sub optimal dose with a sub optimal dose of laquinimod (72.4% activity compared to vehicle) was superior to the optimal dose of fampridine alone (34.5% compared to vehicle).

The combination of laquinimod sub optimal dose with the optimal dose of fampridine (82.8% compared to vehicle) showed a stronger effect than the sub optimal and optimal dose of fampridine alone (24.1% and 34.5% activity, respectively, compared to vehicle).

### Discussion

Safety issues relating to fampridine dosage remain a concern for MS patients, the major concern being the risk of seizures. The inventors have surprising found a treatment regimen for MS (i.e., laquinimod in combination with fampridine) which permits the use of fampridine at a reduced dosage, accompanied by reduced risk of seizures, but results in increased efficacy.

In this study, each compound alone showed a dose dependent inhibition of disease severity. However, the inventors surprisingly found that the combination of fampridine and laquinimod when each was administered at its respective lower dosage (2.5 mg/kg and 5.0 mg/kg, respectively) was highly potent (>70% inhibition), and more potent than fampridine optimal dose.

As indicated in the AMPYRA® Medication Guide, serious side effects may be associated with AMPYRA® use and may follow any deviation from the prescribed regimen, resulting from increased peak serum levels of the drug. It would be appreciated by the person skilled in the art, that since the risk for side effects and specifically seizures is associated with increased peak serum level associated with the currently recommended (optimal) dosage (10 mg dose b.i.d), reduction of the drug dose would reduce the risk associated with increased peak serum levels.

The unexpected results of our study suggest that lower and suboptimal dosages of laquinimod and fampridine, can be used in combination to achieve a superior therapeutic result compared to the optimal dose of fampridine, with reduced risk for side effects associated with fampridine, specifically seizures.

Improving walking ability has a great influence on the quality of life of the patients. As a symptomatic treatment, AMPYRA® is prescribed as an add-on therapy aiming to improve the walking ability of patients which have ambulatory difficulties. These results suggest that the use of fampridine in combination with laquinimod in specific, would allow the reduction of fampridine dose and lead to safer use of fampridine, with improved therapeutic results.

### REFERENCES

1. Alejandro Horga; Xavier Montalban Jun. 4, 2008; Expert Rev Neurother. 2008; 8(5):699-714.
2. Bever and Judge (2009) “Sustained-release fampridine for multiple sclerosis” Expert Opin. Investig. Drugs, 18A(7):1013-1024.
3. Bjartmar C, Fox R I. (2002) “Pathological mechanisms and disease progression of multiple sclerosis: therapeutic implication”, Drugs of Today. 38:7-29.
4. Brex P A et al., (2002) “A longitudinal study of abnormalities on MRI and disability from multiple sclerosis”, N Engl J. Med. Jan. 17, 2002 346(3):158-64.
5. Brod et al. (2000) Annals of Neurology, 47:127-131.
6. Bruck (2011) “Insight into the mechanism of laquinimod action.” J Neurol Sci. 2011 Jul. 15; 306(1-2):173-9.
7. Burton (2008) “4-aminopyridine toxicity with unintentional overdose in four patients with multiple sclerosis.” Neurology, 71:1833-4.
8. Clinical Trials Website, article entitled “Study of Fampridine-ER Tablets in Patients With Multiple Sclerosis”, retrieved Jul. 10, 2012, <http://clinicaltrials.gov/ct2/show?term=fampridine&cond=multiple+sclerosis&phase=2&rank=7>.
9. De Stefano et al. (1999) “Evidence of early axonal damage in patients with multiple sclerosis”, Neurology. 52(Suppl 2):A378.
10. Dunitz, M. Multiple sclerosis therapeutics, Ed. Rudick and Goodkin. London: Taylor & Francis, 1999.
11. Dunitz, M. Multiple sclerosis therapeutics, Ed. Rudick and Goodkin. London: Taylor & Francis, 1999.
12. EMEA Guideline on Clinical Investigation of Medicinal Products for the Treatment of Multiple Sclerosis (CPMP/EWP/561/98 Rev. 1, November 2006).
13. FDA News Release dated Jan. 22, 2012, entitled “FDA Approves Ampyra to Improve Walking in Adults with Multiple Sclerosis”.
14. Frohman et al., (2003) “The utility of MRI in suspected MS: report of the Therapeutics and Technology Assessment Subcommittee of the American Academy of Neurology”, Neurology. Sep. 9, 2003, 61(5):602-11.
15. Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. For Biologics Eval. and Res., Clin./Pharm., November 1999 <http://www.fda.gov/cber/gdlns/metabol.pdf>.
16. Gurevich et al. (2010) “Laquinimod suppress antigen presentation in relapsing-remitting multiple sclerosis: invitro high-throughput gene expression study” (J. Neuroimmunol. 2010 Apr. 15; 221(1-2):87-94. Epub 2010 Mar 27.
17. Guyatt et al. (1985) “The 6-minute walk: a new measure of exercise capacity in patients with chronic heart failure” Can Med Assoc J., 132:919-823.
18. Hla T, Lee M J, Ancellin N, Paik J H, Kluk M J (2001). “Lysophospholipids-receptor revelations”. Science 294 (5548): 1875-8.
19. Hohlfeld et al. (2000) “The neuroprotective effect of inflammation: implications for the therapy of multiple sclerosis”, J. Neuroimmunol. 107:161-166.
20. Kleinschmidt-DeMasters et al. (2005) New England Journal of Medicine, 353:369-379.
21. Langer-Gould et al. (2005) New England Journal of Medicine, 353:369-379.
22. McDonald, (2001) “Guidelines from the International Panel on the Diagnosis of Multiple Sclerosis” Ann. Neurol. 50:121-127.
43. National MS Society Website, retrieved Jul. 10, 2012 <http://www.nationalmssociety.org/for-professionals/researchers/clinical-study-measures/t25-fw/index.aspx>.
23. Neuhaus et al. (2003) “Immunomodulation in multiple sclerosis: from immunosuppression to neuroprotection”, Trends Pharmacol Sci. 24:131-138.
24. Noseworthy et al. (2000) “Multiple sclerosis”, N Engl J. Med. 343:938-952.
25. Polman et al. (2011) “Diagnostic Criteria for Multiple Sclerosis: 2010 Revisions to the McDonald Criteria”, Ann Neural, 69:292-302.
26. Polman et al., (2005) “Diagnostic criteria for multiple sclerosis: 2005 revisions to the McDonald Criteria”, Annals of Neurology, Volume 58 Issue 6, Pages 840-846.
27. Polman et al., (2005) “Treatment with laquinimod reduces development of active MRI lesions in relapsing MS”, Neurology. 64:987-991.
28. Poser et al. (1983) “New Diagnostic Criteria for Multiple Sclerosis: Guidelines for Research Protocols”, Annals of Neurology, March 1983, 13(3):227-230.
29. RTT News Article dated April 12, 11, entitled “Teva Pharma, Active Biotech Post Positive Laquinimod Phase 3 ALLEGRO Results”.
30. Rudick et al. (2006) New England Journal of Medicine, 354:911-923.
31. Rudick, R. (1999) “Disease-Modifying Drugs for Relapsing-Remitting Multiple Sclerosis and Future Directions for Multiple Sclerosis Therapeutics”, Neurotherapeutics. 56:1079-1084.
32. Runström et al. (2002) “Laquinimod (ABR-215062) a candidate drug for treatment of Multiple Sclerosis inhibits the development of experimental autoimmune encephalomyelitis in IFN-β knock-out mice”, (Abstract), Medicon Valley Academy, Malmoe, Sweden.
33. Sandberg-Wollheim et al. (2005) “48-week open safety study with high-dose oral laquinimod in patients”, Mult Scler. 11:S154 (Abstract).
34. Teva Press Release dated Aug. 1, 2011, entitled “Results of Phase III BRAVO Trial Reinforce Unique Profile of Laquinimod for Multiple Sclerosis Treatment”.
35. The Accorda Website, retrieved Jul. 10, 2012, <http://www.acorda.com/Products/Brand.aspx?mname=menu Products>.
36. The Ampyra® Website, retrieved Jul. 10, 2012, <http://ampyra.com/home/?ssva vid=55 db0747ee0a49eca40355313def d3a9>.
37. The National MS Society (USA), The Disease Modifying Drug Brochure, Oct. 19, 2006.
38. U.S. Patent Application Publication 2010-0322900, published Dec. 23, 2010 (Tarcic et al.).
39. U.S. Pat. No. 6,077,851, issued Jun. 20, 2000 (Bjork et al).
44. United Spinal's MS Scene, News Alerts, article entitled “Positive Vote by FDA Advisory Committee for Fampridine-SR”, dated Oct. 16, 2009.
40. Vollmer et al. (2008) “Glatiramer acetate after induction therapy with mitoxantrone in relapsing multiple sclerosis” Multiple Sclerosis, 00:1-8.
41. Yang et al., (2004) “Laquinimod (ABR-215062) suppresses the development of experimental autoimmune encephalomyelitis, modulates the Th1/Th2 balance and induces the Th3 cytokine TGF-β in Lewis rats”, J. Neuroimmunol. 156:3-9.

1. A method of treating a subject afflicted with multiple sclerosis or presenting a clinically isolated syndrome comprising periodically administering to the subject an amount of laquinimod, and an amount of fampridine, wherein the amounts when taken together are effective to treat the subject.

2. The method of claim 1, wherein the amount of laquinimod and the amount of fampridine when administered together is more effective to treat the subject than when each agent at the same amount is administered alone.

3. The method of claim 1, wherein the laquinimod is laquinimod sodium and/or the fampridine is fampridine chloride.

4. (canceled)

5. The method of claim 1, wherein fampridine is administered in a slow release form.

6. The method of claim 1, wherein the laquinimod and/or the fampridine is administered via oral administration.

7. The method of claim 1, wherein the laquinimod and/or the fampridine is administered once daily or twice daily.

8. (canceled)

9. The method of claim 1, wherein the amount laquinimod administered is less than 0.6 mg/day, 0.25-2.0 mg/day, 0.25 mg/day, 0.3 mg/day, 0.5-1.2 mg/day, 0.5 mg/day, 0.6 mg/day, 1.0 mg/day, or 1.2 mg/day.

10-17. (canceled)

18. The method of claim 1, wherein the amount fampridine administered is less than 20 mg/day, 1.0-20 mg/day, 2.5 mg/day, 5-15 mg/day, 5 mg/day, 10 mg/day, 15 mg/day, 1.25 mg b.i.d., 2.5 mg b.i.d., 5 mg b.i.d., 7.5 mg b.i.d.

19-28. (canceled)

29. The method of claim 1, wherein the amount of laquinimod and the amount of fampridine when taken together is effective to alleviate a symptom of multiple sclerosis in the subject.

30-43. (canceled)

44. The method of claim 1, wherein the administration of laquinimod substantially precedes the administration of fampridine.

45. The method of claim 1, wherein the administration of fampridine substantially precedes the administration of laquinimod.

46. The method of claim 44, wherein the subject is receiving laquinimod therapy prior to initiating fampridine therapy.

47. The method of claim 1, further comprising administration of nonsteroidal anti-inflammatory drugs (NSAIDs), salicylates, slow-acting drugs, gold compounds, hydroxychloroquine, sulfasalazine, combinations of slow-acting drugs, corticosteroids, cytotoxic drugs, immunosuppressive drugs and/or antibodies.

48. (canceled)

49. The method of claim 1, wherein each of the amount of laquinimod when taken alone, and the amount of fampridine when taken alone is effective to treat the subject.

50. The method of claim 1, wherein either the amount of laquinimod or when taken alone, the amount of fampridine when taken alone, or each such amount when taken alone is not effective to treat the subject.

51. The method of claim 1, wherein the subject is a human.

52. A package comprising:
a) a first pharmaceutical composition comprising an amount of laquinimod and a pharmaceutically acceptable carrier;
b) a second pharmaceutical composition comprising an amount of fampridine and a pharmaceutically acceptable carrier; and
c) instructions for use of the first and second pharmaceutical compositions together to treat a subject afflicted with multiple sclerosis or presenting a clinically isolated syndrome.

53. (canceled)

54. A pharmaceutical composition comprising an amount of laquinimod and an amount of fampridine wherein the laquinimod and the fampridine are prepared for simultaneous administration or contemporaneous administration.

55-74. (canceled)

75. The method of claim 1, wherein the multiple sclerosis is relapsing multiple sclerosis.

76. The method of claim 1, wherein the relapsing multiple sclerosis is relapsing-remitting multiple sclerosis.

77. (canceled)

78. (canceled)
